(19) United States
(12) Patent Application Publication
(10) Pub. No.: US 2003/0176715 A1
(43) Pub. Date: Sep. 18, 2003

(54) Method for the vapour-phase partial oxidation of aromatic hydrocarbons

(76) Inventors: Peter Reuter, Mannheim (DE); Bernhard Ulrich, Bockenheim (DE); Thomas Heidemann, Weinheim (DE)

Correspondence Address: KEIL & WEINKAUF, 1350 CONNECTICUT AVENUE, N.W., WASHINGTON, DC, 20036, US

(21) Appl. No.: 10/344,515

(22) PCT Filed: Aug. 20, 2001
(86) PCT No.: PCT/EP01/09585

(30) Foreign Application Priority Data
Aug. 21, 2000 (DE) 100 40 818.4

(51) Int. Cl. C07D 307/89; C07C 65/00

(52) U.S. Cl. 549/248; 562/888

(57) ABSTRACT

Aromatic hydrocarbons are partially oxidized in the gas phase over a catalyst at elevated temperature to form carboxylic acids or carboxylic anhydrides in a process in which a gas stream laden with the starting materials is passed through a shell-and-tube reactor whose temperature is controlled by means of one or more separate thermostatting baths conveyed in countercurrent to the starting gas stream, wherein the difference between the temperature of the thermostatting bath in the region of the reactor outlet and the temperature of the product gas stream leaving the reactor is employed to control the selectivity of the gas-phase oxidation.

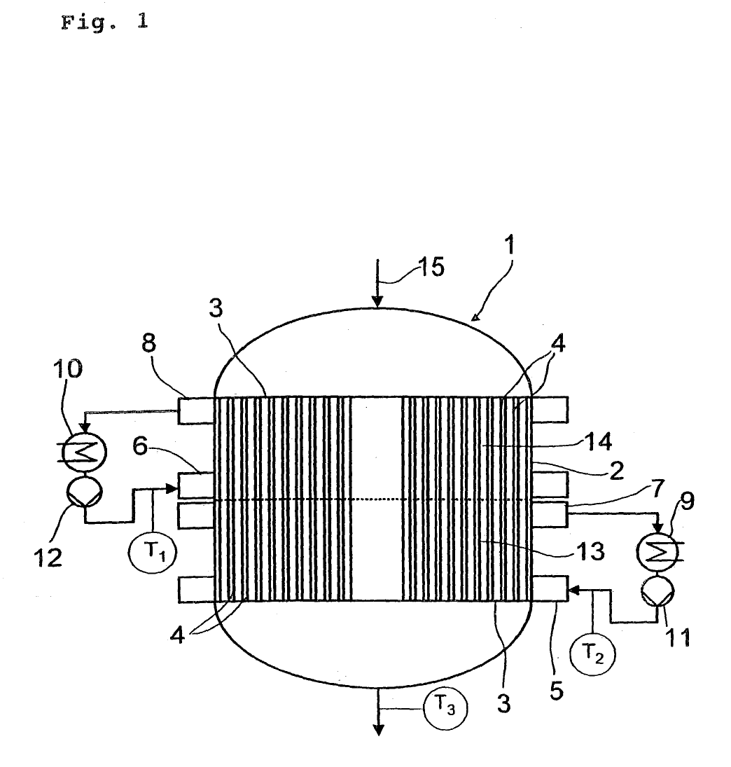

The present invention relates to a process for the gas-phase partial oxidation of aromatic hydrocarbons to form carboxylic acids or carboxylic anhydrides in a shell-and-tube reactor whose temperature is controlled by means of a heat transfer medium which is conveyed through one or more thermostatting baths in countercurrent to the gas stream comprising the reactants.

As is known, a series of carboxylic acids or carboxylic anhydrides, e.g. phthalic anhydride (PA), are prepared industrially by catalytic gas-phase oxidation in fixed-bed reactors, preferably shell-and-tube reactors. In this process, a mixture of a gas comprising molecular oxygen, for example air, and the starting material to be oxidized is generally passed through a large number of tubes located in the reactor. A bed of at least one catalyst is generally present in the tubes. To regulate the temperature, the tubes are surrounded by a heat transfer medium, for example a salt melt. Despite this thermostatting, localized temperature maxima, known as hot spots, in which the temperature is higher than in the remainder of the catalyst bed, can occur. These hot spots give rise to secondary reactions, e.g. total combustion of the starting material, or lead to formation of undesirable by-products which can be separated from the reaction product only with difficulty, if at all.

Excessively high hot spot temperatures generally lead to overoxidation and thus to a severe decrease in the achievable product yield and the operating life of the catalyst. On the other hand, hot spot temperatures which are too low lead to an undesirably high content of underoxidation products, which results in a considerable deterioration in product quality. The hot spot temperature depends on the concentration of starting material in the air stream, on the space velocity of the starting material/air mixture over the catalyst, on the state of aging of the catalyst, on the heat transfer characteristics of the fixed-bed reactor (reactor tube, salt bath) and on the salt bath temperature.

To reduce the hot spots, various measures have been proposed, for example those described in DE 25 46 268 A, EP 286 448 A, DE 29 48 163 A, EP 163 231 A, WO 98/37967, DE 41 09 387 A and DE 198 23 362, for example the zone-wise arrangement of catalysts of differing activity in the catalyst bed in the preparation of PA.

The gas-phase oxidation is controlled industrially via the salt bath temperature. This is determined for each individual reactor under the specific technical conditions by means of analyses of crude and final products. The salt bath temperature is set correctly when only slight overoxidation or total oxidation occurs and the quality of the product is not adversely affected beyond the desired maximum degree by underoxidation products.

However, this method of control is costly and time-consuming. It also has the disadvantage that a significant period of time passes after sampling, analysis and evaluation before an adjustment can be made to the process.

For these reasons, DE 41 09 387 C has proposed, in the preparation of PA, calculating a salt bath temperature to be set at a particular time by means of a formula which relates the instantaneous hot spot temperature and o-xylene concentration and standard values for hot spot and salt bath temperature at a standard o-xylene concentration and a time-dependent apparent activation energy. The formula used is based on linear ageing of the catalyst over time and on the assumption that the optimum salt bath temperature is independent of the volume flow rate of the o-xylene/air mixture. Under these conditions or assumptions, the mathematical expression [T(hot spot)-T(salt bath)]/o-xylene concentration developed in this publication is a parameter proportional to the relevant reaction rate constant. However, such an assumption is not generally applicable, as has been found in industrial practice.

It is an object of the present invention to provide a process for controlling the temperature of shell-and-tube reactors in the catalytic gas-phase partial oxidation of aromatic hydrocarbons which is simple to carry out, is neither time-consuming nor costly and makes it possible to control the oxidation in a simple manner.

We have found that this object is achieved by a process for the catalytic gas-phase partial oxidation of aromatic hydrocarbons to form carboxylic acids or carboxylic anhydrides at elevated temperature, in which a gas stream laden with the starting materials is passed through a shell-and-tube reactor whose temperature is controlled by means of one or more separate thermostatting baths conveyed in countercurrent to the starting gas stream, wherein the difference between the temperature of the thermostatting bath in the region of the reactor outlet and the temperature of the crude product gas stream leaving the reactor is employed to control the selectivity of the gas-phase oxidation. In the following, reference will be made to the thermostatting bath preferred in these gas-phase oxidations, namely a salt bath.

The idea on which the invention is based is to determine the optimum salt bath temperature by measuring the temperature of the thermostatting bath in the region of the reactor outlet and the gas temperature of the product gas stream leaving the reactor (the latter is different from the hot spot temperature). The optimum salt bath temperature can be established without problems from the difference between the measured temperatures.

The basis of this idea is the discovery that the content of by-products (underoxidation products or, if applicable, overoxidation products) found by analysis of the crude product gases from customary processes correlates with the temperature difference between the salt bath temperature at the reactor outlet and the temperature of the crude product gas stream leaving the reactor. If the proportion of underoxidized products is relatively high, the temperature difference is relatively low; on the other hand, if the proportion of underoxidized products is low, the temperature difference is relatively high. Limit values for the temperature difference to be set according to the present invention depend on reactor-specific circumstances and on the gas-phase oxidation concerned.

The thermostatting bath is conveyed in countercurrent to the gas stream comprising the starting materials and has to be cooled to remove heat. This can be achieved in a known manner by means of an internal or external cooling system; cf., for example, Ullmann's Encyclopedia of Industrial Chemistry, 5th Edition, vol. A 20, p. 186. The critical temperature according to the present invention of the thermostatting bath is in both cases in the region of the reactor outlet. In the case of a reactor having external cooling, it is advantageous to employ the temperature of the thermostatting bath entering the reactor in the region of the reactor outlet. For the purposes of the present invention, this means that the temperature is measured at a point after the thermostatting bath has passed through the cooling system and before it enters the reactor. However, the temperature can also be measured after entry into the reactor. This also applies when using two or more thermostatting baths which have separate circuits. The measured value which is decisive for the temperature difference is obtained from the thermostatting bath located nearest the reactor outlet, i.e. in the region of the reactor outlet, cf. the figure explained below.

The temperature difference is preferably selected so that a by-product characteristic of the gas-phase oxidation concerned, generally an underoxidation or overoxidation product, is present in a predetermined concentration range in the product gas stream. The concentration range is dependent on the gas-phase oxidation concerned and on the desired product specifications.

The process of the present invention is preferably employed for preparing phthalic anhydride from o-xylene, naphthalene or mixtures thereof. Phthalide is a characteristic underoxidation product when using o-xylene, and naphthoquinone is a characteristic underoxidation product when using naphthalene.

The process of the present invention can also be used advantageously for the preparation of maleic anhydride from benzene (underoxidation product: furan); pyromellitic anhydride (underoxidation product: 4,5-dimethylphthalic anhydride); benzoic acid from toluene (underoxidation product: benzaldehyde); isophthalic acid from m-xylene (underoxidation product: isophthalaldehyde); and terephthalic acid (underoxidation product: terephthalaldehyde).

In the preparation of PA from o-xylene or naphthalene, the temperature difference is chosen so that it is sufficiently high for the phthalide or naphthoquinone content not to exceed a particular maximum value (e.g. the value laid down in the specification of the PA). Although the phthalide or naphthoquinone content is very low at a high temperature difference, the PA yield is reduced at the same time. For this reason, the temperature difference is in practice selected so that there is a balance between phthalide or naphthoquinone content and PA yield. This is preferably the case when the temperature difference is selected so that the phthalide or naphthoquinone content is in the range from 0.05% to 0.30%, preferably from 0.1% to 0.20%, in each case based on PA. However, depending on the phthalic anhydride specification at the particular site, the upper and lower limit values can also be set at other phthalide or naphthoquinone contents of the product gas stream.

The preferred upper limit for the temperature difference to be set can according to the present invention be established by determining the temperature difference which leads to a phthalide or naphthoquinone content of 0.05%, preferably 0.1%, during running-up of the catalyst.

The preferred lower limit for the temperature difference to be set according to the present invention can be obtained by determining the temperature difference value which leads to a product gas stream having a phthalide or naphthoquinone content of 0.30%, preferably 0.20%.

The procedure for the preparation of products other than PA is analogous.

When the upper and lower limit values for the temperature difference range to be set have been established according to the present invention, the optimum salt bath temperature during further operation of the process of the present invention for the gas-phase oxidation, in particular after reaching a standard loading, can be set without analysis of the crude product gas stream by ensuring that the temperature difference is between the limit values determined.

Oxidic supported catalysts are suitable as catalysts. For the preparation of phthalic anhydride by gas-phase oxidation of o-xylene or naphthalene, use is made of spherical, ring-shaped or dish-shaped supports comprising a silicate, silicon carbide, porcelain, aluminum oxide, magnesium oxide, tin dioxide, rutile, aluminum silicate, magnesium silicate (steatite), zirconium silicate or cerium silicate or a mixture thereof. In general, the catalytically active constituent employed is titanium dioxide, in particular in the form of its anatase modification, together with vanadium pentoxide. The catalytically active composition may further comprise small amounts of many other oxidic compounds which act as promoters to influence the activity and selectivity of the catalyst, for example by reducing or increasing its activity. Examples of such promoters are the alkali metal oxides, thallium(I) oxide, aluminum oxide, zirconium oxide, iron oxide, nickel oxide, cobalt oxide, manganese oxide, tin oxide, silver oxide, copper oxide, chromium oxide, molybdenum oxide, tungsten oxide, iridium oxide, tantalum oxide, niobium oxide, arsenic oxide, antimony oxide, cerium oxide and phosphorus pentoxide. For example, the alkali metal oxides act as promoters which reduce the activity and increase the selectivity, while oxidic phosphorus compounds, in particular phosphorus pentoxide, increase the activity of the catalyst but reduce its selectivity. Examples of catalysts which can be used are described, for example, in DE 25 10 994, DE 25 47 624, DE 29 14 683, DE 25 46 267, DE 40 13 051, WO 98/37965 and WO 98/37967.

Catalysts which have been found to be particularly useful are coated catalysts in which the catalytically active composition is applied in the form of a shell to the support (cf., for example, DE 16 42 938 A, DE 17 69 998 A and WO 98/37967).

Catalysts for the other products mentioned above are V₂O₅/MoO₃ (maleic anhydride), V₂O₅ (pyromellitic anhydride, cf. DE 1593536), cobalt naphthenate (benzoic acid) and Co—Mn—Br catalysts (isophthalic and terephthalic acid).

To carry out the reaction, the catalysts are introduced into the tubes of a shell-and-tube reactor. The reaction gas is passed at elevated temperature and at superatmospheric pressure over the catalyst bed prepared in this way. The reaction conditions are dependent on the desired product and the reaction circumstances, e.g. catalyst, loading with starting material, etc., and can be taken from customary reference works, e.g. Ullmann's Encyclopedia of Industrial Chemistry, 5th Edition, VCH Verlagsgesellschaft. The preparation of PA from o-xylene is generally carried out at from 300 to 450° C., preferably from 320 to 420° C. and particularly preferably from 340 to 400° C., and at a gauge pressure of from 0.1 to 2.5 bar, preferably from 0.3 to 1.5 bar, at a space velocity of from 750 to 5000 h⁻¹.

The reaction gas fed to the catalyst is generally prepared by mixing a gas which comprises molecular oxygen and may further comprise suitable reaction moderators and/or diluents, e.g. steam, carbon dioxide and/or nitrogen, with the aromatic hydrocarbon to be oxidized. The reaction gas generally contains from 1 to 100 mol %, preferably from 2 to 50 mol % and particularly preferably from 10 to 30 mol %, of oxygen. In general, the reaction gas is loaded with from 5 to 120 g, preferably from 60 to 120 g and particularly preferably from 80 to 115 g, of the aromatic hydrocarbon to be oxidized per standard m³ of gas.

It has been found to be advantageous to arrange two or more catalysts of differing activity in zones in the catalyst bed or in two or more separate reactors, with the catalysts generally being arranged so that the reaction gas mixture comes into contact with the less active catalyst first (first reaction zone) and only subsequently with the more active catalyst (second reaction zone). The first reaction zone closest to the reaction gas inlet generally makes up from 30 to 80% of the total catalyst volume and can be thermostatted to a reaction temperature which is from 1 to 20° C. higher, preferably from 1 to 10° C. higher and in particular from 2 to 8° C. higher, than that of the second reaction zone. Alternatively, both reaction zones can have the same temperature. In the preparation of PA, a vanadium pentoxide/titanium dioxide catalyst doped with alkali metal oxides is generally used in the first reaction zone and a vanadium pentoxide/titanium dioxide catalyst doped with a smaller amount of alkali metal oxides and/or with phosphorus compounds is used in the second reaction zone.

The reaction is generally controlled by means of the temperature setting so that the major part of the aromatic hydrocarbon present in the reaction gas is reacted in maximum yield in the first zone.

In a reactor configuration having two or more zones, the salt bath temperature of the salt bath closest to the reactor outlet or of the second reactor is particularly preferably adjusted without changing the salt bath temperature of the salt bath closest to the reactor inlet or of the first reactor.

The following examples illustrate the invention without restricting its scope.

### EXAMPLE OF CATALYST PRODUCTION

Production of Catalysts I and II for the Preparation of PA

Catalyst I (two batches of this catalyst I were produced): 50 kg of steatite (magnesium silicate) rings having an external diameter of 8 mm, a length of 6 mm and a wall thickness of 1.5 mm were heated to 160° C. in a coating drum and sprayed with a suspension comprising 28.6 kg of anatase having a BET surface area of 20 m²/g, 4.11 kg of vanadyl oxalate, 1.03 kg of antimony trioxide, 0.179 kg of ammonium dihydrogen phosphate, 0.184 kg of caesium sulfate, 44.1 kg of water and 9.14 kg of formamide until the weight of the applied layer after calcination at 450° C. was 10.5% of the total weight of the finished catalyst.

The catalytically active composition applied in this way, i.e. the catalyst shell, comprised 0.15% by weight of phosphorus (calculated as P), 7.5% by weight of vanadium (calculated as V₂O₅), 3.2% by weight of antimony (calculated as Sb₂O₃), 0.4% by weight of caesium (calculated as Cs) and 89.05% by weight of titanium dioxide.

Catalyst II: 50 kg of steatite (magnesium silicate) rings having an external diameter of 8 mm, a length of 6 mm and a wall thickness of 1.5 mm were heated to 160° C. in a coating drum and sprayed with a suspension comprising 28.6 kg of anatase having a BET surface area of 11 m²/g, 3.84 kg of vanadyl oxalate, 0.80 kg of antimony trioxide, 0.597 kg of ammonium dihydrogen phosphate, 44.1 kg of water and 9.14 kg of formamide until the weight of the applied layer after calcination at 450° C. was 12.5% of the total weight of the finished catalyst.

The catalytically active composition applied in this way, i.e. the catalyst shell, comprised 0.50% by weight of phosphorus (calculated as P), 7.0% by weight of vanadium (calculated as V₂O₅), 2.5% by weight of antimony (calculated as Sb₂O₃) and 90.05% by weight of titanium dioxide.

### PROCESS EXAMPLES AND COMPARATIVE EXAMPLES

The examples are described below with reference to the figure. This schematically shows a cross section through a PA reactor.

Preparation of PA

The reactor 1 has a cylindrical section 2 which is bounded by two tube plates 3. In the cylindrical section, a large number (in the present example 100) of cylindrical iron tubes 4 having an internal diameter of 25 mm extend between the tube plates 3. 1.30 m of the catalyst II and subsequently (above the catalyst II) 1.60 m of the catalyst I were introduced into each of the 3.85 m long iron tubes 4. To regulate the temperature, the iron tubes were surrounded by a salt melt which was divided into two separate salt baths 13 and 14.

Each of the salt baths was circulated by means of the pumps 11 and 12. Entry into the salt baths 13 and 14 was via the ports 5 and 6, respectively, and the exit was via the ports 7 and 8, respectively. After leaving the reactor, the salt baths are conveyed through the heat exchangers 9 and 10, respectively. The measurement points for determining the temperature difference were T2 at the inlet for the lower salt bath 13 and T3 at the outlet for the product gas stream. In addition, the temperature was also determined at the point at which the upper salt bath 14 entered the reactor (measurement point T1).

The reactor was supplied with the starting gas stream 15. 4.0 standard m³ of air laden with from 50 to about 80 g of 98.5% strength by weight o-xylene per standard m³ of air were passed per hour and per tube, from the top downward through the tubes 4. The results summarized in the following table were obtained (day=day of operation from the first running-up of the catalyst; gas outlet T=crude product gas temperature at the end of the reactor (measurement point T3); SBT top=temperature of the salt melt at the point at which it enters the salt bath 14 closest to the reactor inlet (measurement point T 1); SBT bottom=temperature of the salt melt at the point at which it enters the salt bath 13 closest to the reactor outlet (measurement point T 2); PHDE content=phthalide content of the crude product gas, relative to phthalic anhydride; temperature difference difference between SBT bottom and gas outlet T).

Under conditions where the upper PHDE limit is 0.30% and the lower PHDE limit is 0.05%, an optimum operating state of the catalyst is achieved when the temperature difference is from 13° C. to 15° C. If the temperature difference is below 13° C., a PA of unsatisfactory quality (PHDE content too high) is obtained, while if the temperature difference is more than 15° C., the catalyst is subjected to unnecessary thermal stress and the achievable PA yield decreases.

| | gas | | SBT | PHDE- | | | temp.- |
|---|---|---|---|---|---|---|---|
| | outlet | SBT | bot- | con- | load | PA- | differ- |
| | temp. | top | tom | tent | (g/Nm³- | yield | ence |
| day | (° C.) | (° C.) | (° C.) | (%) | Luft) | (m/m %) | (° C.) |
| **114 | 349 | 365 | 365 | 0.04 | 70 | 111.0 | 16 |
| 117 | 348 | 363 | 363 | 0.07 | 75 | 111.6 | 15 |
| 126 | 348 | 361 | 361 | 0.12 | 80 | 111.9 | 13 |
| 132 | 347 | 361 | 361 | 0.06 | 80 | 111.7 | 14 |
| 133 | 345 | 360 | 360 | 0.09 | 80 | 111.9 | 15 |
| 136 | 345 | 360 | 360 | 0.09 | 80 | 111.7 | 15 |
| 137 | 345 | 359 | 359 | 0.11 | 80 | 111.9 | 14 |
| 138 | 345 | 358 | 358 | 0.12 | 80 | 111.8 | 13 |
| 139 | 344 | 357 | 357 | 0.13 | 80 | 111.7 | 13 |
| 140 | 343 | 356 | 356 | 0.14 | 80 | 111.7 | 13 |
| 143 | 342 | 355 | 355 | 0.16 | 80 | 112.3 | 13 |
| 144 | 341 | 354 | 354 | 0.20 | 80 | 112.5 | 13 |
| *150 | 341 | 353 | 353 | 0.31 | 80 | 122.8 | 12 |
| 201 | 346 | 362 | 360 | 0.15 | 80 | 112.7 | 14 |
| 206 | 347 | 362 | 360 | 0.16 | 80 | 112.8 | 13 |
| 208 | 347 | 362 | 361 | 0.15 | 80 | 112.5 | 14 |
| 215 | 346 | 361 | 361 | 0.14 | 80 | 112.3 | 15 |
| 224 | 348 | 361 | 361 | 0.15 | 80 | 112.4 | 13 |
| 228 | 346 | 361 | 361 | 0.14 | 80 | 112.4 | 15 |
| 240 | 352 | 365 | 365 | 0.09 | 70 | 112.1 | 13 |
| 241 | 350 | 365 | 365 | 0.09 | 70 | 112.3 | 15 |
| 242 | 340 | 365 | 355 | 0.17 | 80 | 112.6 | 15 |
| 244 | 342 | 365 | 355 | 0.18 | 80 | 113.4 | 13 |
| *247 | 333 | 365 | 345 | 0.31 | 80 | 113.5 | 12 |
| *248 | 333 | 365 | 345 | 0.35 | 80 | 114 | 12 |
| *250 | 330 | 365 | 340 | 0.53 | 80 | 114.3 | 10 |
| *251 | 329 | 365 | 340 | 0.51 | 80 | 113.3 | 11 |
| *255 | 328 | 365 | 340 | 0.30 | 80 | 113.4 | 12 |
| *261 | 324 | 365 | 335 | 0.68 | 80 | 114.6 | 11 |
| *262 | 319 | 365 | 330 | 0.68 | 80 | 114.9 | 11 |
| *266 | 319 | 366 | 330 | 0.73 | 80 | 114.6 | 11 |
| 384 | 346 | 361 | 361 | 0.06 | 80 | 111.6 | 15 |
| 390 | 340 | 361 | 355 | 0.09 | 80 | 112.0 | 15 |
| **396 | 351 | 371 | 371 | 0.00 | 50 | 110.5 | 20 |
| **403 | 350 | 369 | 369 | 0.00 | 60 | 111.1 | 19 |
| **406 | 351 | 369 | 369 | 0.01 | 60 | 110.4 | 18 |
| **417 | 347 | 364 | 364 | 0.02 | 70 | 111.6 | 17 |
| *423 | 346 | 364 | 364 | 0.02 | 70 | 110.9 | 18 |
| **428 | 344 | 362 | 362 | 0.03 | 80 | 111.0 | 18 |
| **429 | 345 | 362 | 362 | 0.03 | 80 | 111.3 | 17 |
| **437 | 345 | 362 | 362 | 0.03 | 80 | 111.2 | 17 |
| **449 | 344 | 360 | 360 | 0.04 | 80 | 111.9 | 16 |
| 450 | 344 | 359 | 359 | 0.06 | 80 | 112.0 | 15 |
| 466 | 344 | 358 | 358 | 0.09 | 80 | 112.3 | 14 |
| 469 | 342 | 358 | 355 | 0.12 | 80 | 112.8 | 13 |

*PHDE-content >0.30% => catalyst is too inactive, SBT must be raised;
**PHDE-content <0.05% in the gasphase => catalyst temperature is too high, SBT can be lowered.

The table shows the variation of the phthalide content with the temperature difference. In case the phthalide content is above the desired value (e.g. >0.30%), the catalyst is too inactive and the salt bath temperature must be raised. In case the phthalide content is below the desired value, the catalyst is operated at a temperature too high and the salt bath temperature must be lowered.

We claim:

1. A process for the catalytic gas-phase partial oxidation of aromatic hydrocarbons to form carboxylic acids or carboxylic anhydrides at elevated temperature, in which a gas stream laden with the starting materials is passed through a shell-and-tube reactor whose temperature is controlled by means of one or more separate thermostatting baths conveyed in countercurrent to the starting gas stream, wherein the difference between the temperature of the thermostatting bath in the region of the reactor outlet and the temperature of the product gas stream leaving the reactor is employed to control the selectivity of the gas-phase oxidation.

2. A process as claimed in claim 1, wherein the temperature difference is selected so that a by-product characteristic of the gas-phase oxidation concerned is present in a predetermined concentration range in the product gas stream.

3. A process as claimed in claim 1 or 2, wherein the temperature difference in the gas-phase partial oxidation of o-xylene to phthalic anhydride is selected so that the phthalide content of the phthalic anhydride is in the range from 0.05% to 0.3%, based on phthalic anhydride.

4. A process as claimed in claim 3, wherein the temperature difference is selected so that the phthalide content of the product gas stream is in the range from 0.1% to 0.2%, based on phthalic anhydride.

5. A process as claimed in claim 1 or 2, wherein the temperature difference in the gas-phase partial oxidation of naphthalene to phthalic anhydride is selected so that the naphthoquinone content of the product gas stream is in the range from 0.05 to 0.3% by weight, based on phthalic anhydride.

6. A process as claimed in claim 5, wherein the temperature difference is selected so that the naphthoquinone content of the product gas stream is in the range from 0.1 to 0.2% by weight, based on phthalic anhydride.

7. A process as claimed in any of claims 3 to 6, wherein the reaction is carried out over supported vanadium pentoxide/titanium dioxide catalysts.

8. A process as claimed in any of claims 3 to 7, wherein the reaction is carried out at from 300° C. to 450° C.

9. A process as claimed in any of claims 3 to 8, wherein the reaction is carried out at a gauge pressure of from 0.1 to 2.5 bar.
